SEQUENCE LISTING (10 sequences)

SEQ ID NO: 1 - protein, 605 aa, Homo sapiens
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 2 - protein, 274 aa, Homo sapiens
MVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 3 - protein, 533 aa, Homo sapiens
MAFANLRKVLISDSLDPCCRKILQDGGLQVVEKQNLSKEELIAELQDCEGLIVRSATKVTADVINAAEKLQVVGRAGTGVDNVDLEAATRKGILVMNTPNGNSLSAAELTCGMIMCLARQIPQATASMKDGKWERKKFMGTELNGKTLGILGLGRIGREVATRMQSFGMKTIGYDPIISPEVSASFGVQQLPLEEIWPLCDFITVHTPLLPSTTGLLNDNTFAQCKKGVRVVNCARGGIVDEGALLRALQSGQCAGAALDVFTEEPPRDRALVDHENVISCPHLGASTKEAQSRCGEEIAVQFVDMVKGKSLTGVVNAQALTSAFSPHTKPWIGLAEALGTLMRAWAGSPKGTIQVITQGTSLKNAGNCLSPAVIVGLLKEASKQADVNLVNAKLLVKEAGLNVTTSHSPAAPGEQGFGECLLAVALAGAPYQAVGLVQGTTPVLQGLNGAVFRPEVPLRRDLPLLLFRTQTSDPAMLPTMIGLLAEAGVRLLSYQTSLVSDGETWHVMGISSLLPSLEAWKQHVTEAFQFHF

SEQ ID NO: 4 - protein, 288 aa, Homo sapiens
MERPQPDSMPQDLSEALKEATKEVHTQAENAEFMRNFQKGQVTRDGFKLVMASLYHIYVALEEEIERNKESPVFAPVYFPEELHRKAALEQDLAFWYGPRWQEVIPYTPAMQRYVKRLHEVGRTEPELLVAHAYTRYLGDLSGGQVLKKIAQKALDLPSSGEGLAFFTFPNIASATKFKQLYRSRMNSLEMTPAVRQRVIEEAKTAFLLNIQLFEELQELLTHDTKDQSPSRAPGLRQRASNKVQDSAPVETPRGKPPLNTRSQAPLLRWVLTLSFLVATVAVGLYAM

SEQ ID NO: 5 - protein, 501 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL

SEQ ID NO: 6 - protein, 137 aa, Homo sapiens
MGLRAGGTLGRAGAGRGAPEGPGPSGGAQGGSIHSGRIAAVHNVPLSVLIRPLPSVLDPAKVQSLVDTIREDPDSVPPIDVLWIKGAQGGDYFYSFGGCHRYAAYQQLQRETIPAKLVQSTLSDLRVYLGASTPDLQ

SEQ ID NO: 7 - protein, 317 aa, Homo sapiens
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 8 - protein, 190 aa, Homo sapiens
MAFIAKSFYDLSAISLDGEKVDFNTFRGRAVLIENVASLUGTTTRDFTQLNELQCRFPRRLVVLGFPCNQFGHQENCQNEEILNSLKYVRPGGGYQPTFTLVQKCEVNGQNEHPVFAYLKDKLPYPYDDPFSLMTDPKLIIWSPVRRSDVAWNFEKFLIGPEGEPFRRYSRTFPTINIEPDIKRLLKVAI

SEQ ID NO: 9 - protein, 226 aa, Homo sapiens
MARLLQASCLLSLLLAGFVSQSRGQEKSKMDCHGGISGTIYEYGALTIDGEEYIPFKQYAGKYVLFVNVASYUGLTGQYIELNALQEELAPFGLVILGFPCNQFGKQEPGENSEILPTLKYVRPGGGFVPNFQLFEKGDVNGEKEQKFYTFLKNSCPPTSELLGTSDRLFWEPMKVHDIRWNFEKFLVGPDGIPIMRWHHRTTVSNVKMDILSYMRRQAALGVKRK

SEQ ID NO: 10 - protein, 187 aa, Homo sapiens
MVAATVAAAWLLLWAAACAQQEQDFYDFKAVNIRGKLVSLEKYRGSVSLVVNVASECGFTDQHYRALQQLQRDLGPHHFNVLAFPCNQFGQQEPDSNKEIESFARRTYSVSFPMFSKIAVTGTGAHPAFKYLAQTSGKEPTWNFWKYLVAPDGKVVGAWDPTVSVEEVRPQITALVRKLILLKREDL

(19) United States
(12) Patent Application Publication
(10) Pub. No.: US 2023/0220481 A1
(43) Pub. Date: Jul. 13, 2023

(54) Method for Determining Sensitivity to 2,2'-Dithio-bis-Ethane Sulfonate

(71) Applicant: Lantern Pharma Inc., Dallas, TX (US)

(72) Inventors: Aditya Kulkarni, Dallas, TX (US); Kishor Bhatia, Ellicott City, MD (US)

(21) Appl. No.: 17/811,812

(22) Filed: Jul. 11, 2022

Related U.S. Application Data
(63) Continuation of application No. PCT/US2021/012994, filed on Jan. 11, 2021.
(60) Provisional application No. 62/959,615, filed on Jan. 10, 2020.

(51) Int. Cl. C12Q 1/6886

(52) U.S. Cl. CPC C12Q 1/6886; C12Q 2600/158; C12Q 2600/106; C12Q 1/6869

(57) ABSTRACT

A method for treating cancer in a patient in which a sample of the tumor or cancer is obtained from the patient and screened for responsiveness to 2,2′-dithio-bis-ethane sulfonate analogs. The sample is treated with 2,2′-dithio-bis-ethane sulfonate analogs to determine whether one or more of a plurality of biomarkers will be expressed therein.

### TECHNICAL FIELD

This application relates to determining the presence and/or level of biomarkers for detecting sensitivity of a patient to 2,2′-Dithio-bis-Ethane Sulfonate as part of a cancer treatment. The application relates to methods to detect the expression levels of genes encoding biomarkers in cancer patients and to predict the responsiveness of cancer patients to disodium 2,2′-dithio-bis-ethane sulfonate as part of a cancer therapy.

### INCORPORATION BY REFERENCE OF SEQUENCE LISTING

The Sequence Listing titled 197585010046.XML, which was created on Mar. 30, 2023, and is 13 kilobytes in size, is hereby incorporated by reference in its entirety.

### BACKGROUND

Cancer chemotherapy has been an expanding area of scientific endeavor and has been a critical component of cancer treatment along with surgery and radiation therapy. Where chemotherapy was once accepted only as a means to extend survival time for those patients diagnosed as incurable by surgery and/or radiation therapy, it is now a recognized modality of treatment in nearly all variations of cancer.

Modern cancer chemotherapy typically involves a combination of two or more different drugs, and the advances in technology and medical knowledge have greatly improved a patient's chances of recovery in many forms of cancer. The role of chemotherapy agents in cancer therapy varies widely depending upon the form of cancer. For example, chemotherapy is often the primary course of therapy in cancers of the ovary, testis, breast, bladder, and others, in leukemias and lymphomas, and is generally employed in combination with radiation therapy in the treatment of a large number of sarcomas, melanomas, myelomas, and others.

Chemotherapeutic agents are classified into a number of diverse groups. The vast majority of these agents act as cytotoxic drugs, and each member of a specific group is postulated to typically exert its cytotoxic effects through a similar biological mechanism. A complete understanding of the biological and biochemical mechanisms of action of antineoplastic drugs are not fully known.

Accordingly, there is always a need for methods that can determine or predict whether a cancer will be responsive to a chemotherapeutic agent, including 2,2′-dithio-bis-ethane sulfonate or its analogs.

### SUMMARY

This application discloses a method for treating cancer in a patient in which a sample of the tumor or cancer is obtained from the patient and screened for responsiveness to 2,2′-dithio-bis-ethane sulfonate or its analogs. The sample is treated with 2,2′-dithio-bis-ethane sulfonate or its analogs to determine whether one or more of a plurality of biomarkers will be expressed therein. The biomarkers or plurality of biomarkers can be nuclear factor erythroid 2-related factor 2 (NRF2) or other biomarkers. The sample indicates the patient may be responsive to 2,2′-dithio-bis-ethane sulfonate or its analogs if the expression level of NRF2 (and/or other biomarkers) is greater than 1 time the non-cancer cells or a reference level.

One aspect includes a method in which a plurality of biomarkers further includes one selected from the group consisting of NQO1 (SEQ ID NO: 2), PHGDH (SEQ ID NO: 3), HMOX1 (SEQ ID NO: 4), SLC7A11 (SEQ ID NO: 5), SRXN1 (SEQ ID NO: 6), SOX2(SEQ ID NO: 7), GPX2 (SEQ ID NO: 8), GPX3 (SEQ ID NO: 9), and GPX7(SEQ ID NO: 10. The non-cancer cells define the reference level for the biomarkers.

The cancer treatments comprise the administration of one or more chemotherapy agents selected from platinum complexes and taxanes. A cancer treatment includes radiation treatment.

A method of testing a tumor sample of a patient having a known cancer type in which the patient is resistant to one or more cancer therapies and has an unknown responsiveness to 2,2′-dithio-bis-ethane sulfonate or its analogs. The method includes contacting the sample with 2,2′-dithio-bis-ethane sulfonate or its analogs; contacting the sample with a device comprising (a) a first single-stranded nucleic acid molecules capable of specifically hybridizing with the nucleotides of a plurality of biomarkers, wherein a first biomarker is NFR2; and (b) a second single-stranded nucleic acid molecules capable of specifically hybridizing with the nucleotides of a plurality of biomarkers selected from NQO1 (SEQ ID NO: 2), PHGDH (SEQ ID NO: 3), HMOX1 (SEQ ID NO: 4), SLC7A11 (SEQ ID NO: 5), SRXN1 (SEQ ID NO: 6), SOX2(SEQ ID NO: 7), GPX2 (SEQ ID NO: 8), GPX3 (SEQ ID NO: 9), and GPX7(SEQ ID NO: 10); detecting a level of expression of the plurality of biomarkers; and administering 2,2′-dithio-bis-ethane sulfonate or its analogs to the patient, wherein the patient has been determined to be responsive to 2,2′-dithio-bis-ethane sulfonate or its analogs from the expression of the biomarkers from reference levels.

As will be apparent, features and characteristics of one aspect of the application are applicable to many other aspects of the application. The invention is hereinafter described by way of the following non-limiting Examples and with reference to the accompanying figures.

### BRIEF DESCRIPTION OF THE FIGURES

FIG. 1 shows the induction of nuclear expression of Nrf2 in cells treated with t-BHQ 30 μM and cells treated with 2,2′-dithio-bis-ethane sulfonate.

### SEQUENCE LISTING

SEQ ID NO:1—amino acid sequence of NRF2 (NFE2L2),

SEQ ID NO:2—amino acid sequence of NQ01,

SEQ ID NO:3—amino acid sequence of PHGDH,

SEQ ID NO:4—amino acid sequence of HMOX1,

SEQ ID NO:5—amino acid sequence of SLC7A11,

SEQ ID NO:6—amino acid sequence of SRXN1,

SEQ ID NO:7—amino acid sequence of SOX2,

SEQ ID NO:8—amino acid sequence of GPX2,

SEQ ID NO:9—amino acid sequence of GPX3, and

SEQ ID NO: 10—amino acid sequence of and GPX7.

### DEFINITIONS

Unless specifically defined otherwise, all technical and scientific terms used herein shall be taken to have the same meaning as commonly understood by one of ordinary skill in the art (e.g., in cell culture, molecular genetics, immunology, immunohistochemistry, protein chemistry, and biochemistry).

Amino acid sequence aligned with the amino acid sequence set out in SEQ ID NO: X (when referring to a variant polypeptide) means that the variant amino acid sequence and the amino acid sequence set out in SEQ ID NO: X are aligned by a suitable method which allows comparison of the sequences with each other and identifications of the positions in the amino acid sequence of the variant wherein either the same amino acid is present (identical position), or another amino acid is present (substitution), or one or more extra amino acids are present (insertion or extension) or no amino acid is present (deletion or truncation) if compared with the amino acid sequence set out in SEQ ID NO: X.

The term “antibody” as used herein includes intact molecules as well as molecules comprising or consisting of fragments thereof, such as, for example Fab, F(ab′)2, Fv and scFv, as well as engineered variants including diabodies, triabodies, mini-bodies and single-domain antibodies which are capable of binding an epitopic determinant. Thus, antibodies may exist as intact immunoglobulins, or as modifications in a variety of forms.

The term “biomarker” refers to any molecule, such as a gene, gene transcript (for example mRNA), peptide or protein or fragment thereof produced by a subject which is useful in differentiating subjects to predict the responsiveness of patients to treatments including disodium 2,2′-dithio-bis-ethane sulfonate or its analogs. A biomarker that is differentially present (i.e., increased or decreased) in a biological sample from a subject or a group of subjects has a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (e.g., a p-value less than 0.05 and/or a q-value of less than 0.10 as determined using either Welch's T-test or Wilcoxon's rank-sum Test).

The terms “cancer” and “cancerous” refer to or describe the physiological condition in mammals (e.g., humans) that is typically characterized by unregulated cell proliferation. Examples of cancer include, but are not limited to, prostate cancer, ovarian cancer (e.g., ovarian adenocarcinoma or embryonal carcinoma), liver cancer (e.g., hepatocellular carcinoma (HCC) or hepatoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), breast cancer (e.g., medullary carcinoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. The term cancer includes solid tumors (e.g., prostate cancer, ovarian cancer, or hepatocellular carcinoma (HCC)) and hematological cancers.

The term “diagnosis”, and variants thereof such as, but not limited to, “diagnose”, “diagnosed” or “diagnosing” shall not be limited to a primary diagnosis of a clinical state, but should be taken to include diagnosis of recurrent disease.

The term “subject” refers to any animal that may develop cancer and includes animals such as mammals, e.g., humans, or non-human mammals such as cats and dogs, laboratory animals such as mice, rats, rabbits or guinea pigs, and livestock animals. In a preferred embodiment, the subject is a human.

The “sample” may be of any suitable type and may refer, e.g., to a material in which the presence or level of biomarkers can be detected. Preferably, the sample is obtained from the subject so that the detection of the presence and/or level of biomarkers may be performed in vitro. Alternatively, the presence and/or level of biomarkers can be detected in vivo. The sample can be used as obtained directly from the source or following at least one step of (partial) purification. The sample can be prepared in any convenient medium which does not interfere with the method of the invention. Typically, the sample is an aqueous solution, biological fluid, cells or tissue. Preferably, the sample is blood, plasma, serum, urine, platelets or other material. Pre-treatment may involve, for example, preparing plasma from blood, diluting viscous fluids, and the like. Methods of treatment can involve filtration, distillation, separation, concentration, inactivation of interfering components, and the addition of reagents. The selection and pre-treatment of biological samples prior to testing is well known in the art and need not be described further.

The terms “expression level” and “level of expression,” as used herein, refer to the amount of a gene product in a cell, tissue, biological sample, organism, or patient, e.g., amounts of DNA, RNA (e.g. messenger RNA (mRNA)), or proteins corresponding to a given gene.

The term “healthy individual” shall be taken to mean an individual who is known not to suffer from cancer, such knowledge being derived from clinical data on the individual, including, but not limited to, a different diagnostic assay to that described herein.

A “reference level” means a level of the compound of the present invention or additional biomarker(s) that is indicative of a particular disease state, phenotype, or lack thereof, as well as combinations of disease states, phenotypes, or lack thereof.

A “reference sample” refers to a sample containing reference level of a biomarker. For example, a reference sample can be obtained from a subject that does not have a particular disease, disease state or phenotype, such as cancer or acute injury.

### DETAILED DESCRIPTION

The application includes methods for detecting a patient having cancer, e.g., a patient having cancer that is resistant to one or more cancer therapies other than 2,2′-dithio-bis-ethane sulfonate or its analogs (e.g., a patient with lung cancer, prostate cancer, ovarian cancer, or hepatocellular carcinoma (HCC) that is resistant to one or more cancer therapies other than 2,2′-dithio-bis-ethane sulfonate or its analogs alone or in combination with one or more therapies), and for determining responsiveness of a cancer patient (e.g., a patient with lung cancer, prostate cancer, ovarian cancer, or HCC) to treatment with 2,2′-dithio-bis-ethane sulfonate or its analogs, alone or in combination with other therapies. This application also features methods of treating cancer in a patient in need thereof (e.g., a patient with lung cancer, prostate cancer, ovarian cancer, or HCC or a treatment resistant form thereof) that include administering 2,2′-dithio-bis-ethane sulfonate or its analogs to the patient, in which the patient is or has been determined to be responsive to 2,2′-dithio-bis-ethane sulfonate or its analogs according to the diagnostic methods described herein. Responsiveness may be a protective effect (protect against effects of cancer drugs) shown by 2,2′-dithio-bis-ethane sulfonate or its analogs and/or anticancer activity. Responsiveness is marked by one or a plurality of biomarkers.

One embodiment includes a method of testing a tumor sample of a patient having a known cancer type having known or unknown responsiveness to 2,2′-dithio-bis-ethane sulfonate or its analogs having the steps of (a) contacting the sample with 2,2′-dithio-bis-ethane sulfonate or its analogs, (b) contacting the sample, after contact with 2,2′-dithio-bis-ethane sulfonate or its analogs, with a device comprising: i) single-stranded nucleic acid molecules capable of specifically hybridizing with the nucleotides of a plurality of biomarkers of sensitivity selected from the biomarkers of NFR2/NFE2L2 (SEQ ID NO: 1); and/or ii) single-stranded nucleic acid molecules capable of specifically hybridizing with the nucleotides of a plurality of biomarkers of resistance selected from the biomarkers of NQO1 (SEQ ID NO: 2), PHGDH (SEQ ID NO: 3), HMOX1 (SEQ ID NO: 4), SLC7A11 (SEQ ID NO: 5), SRXN1 (SEQ ID NO: 6), SOX2(SEQ ID NO: 7), GPX2 (SEQ ID NO: 8), GPX3 (SEQ ID NO: 9), and GPX7(SEQ ID NO: 10); (c) detecting a level of expression of the plurality of biomarkers. When the biomarkers are above the typical amounts in non-tumor cells or reference cells, the patient can be responsive to a treatment including 2,2′-dithio-bis-ethane sulfonate or its analogs.

In some examples, the cancer is selected from the group consisting of prostate cancer, ovarian cancer, hepatocellular carcinoma (HCC), cervical cancer, renal cell carcinoma (RCC), esophageal cancer, melanoma, glioma, pancreatic cancer, gastrointestinal stromal tumors (GIST), sarcoma, estrogen receptor-positive (ERpos) breast cancer, non-small cell lung carcinoma (NSCLC), colon cancer, bladder cancer, squamous cell carcinoma of the head and neck (SCCHN), acute myelogenous leukemia (AML), acute lympho-blastic leukemia (ALL), chronic lymphocytic leukemia (CLL), myelodysplastic syndrome (MDS), chronic myelogenous leukemia-chronic phase (CMLCP), diffuse large B-cell lymphoma (DLBCL), cutaneous T-cell lymphoma (CTCL), peripheral T-cell lymphoma (PTCL), and Hodgkin's lymphoma.

A patient to be treated or tested for responsiveness to a treatment (e.g., 2,2′-dithio-bis-ethane sulfonate) according to the methods described herein may be one who has been diagnosed with a cancer, such as those described herein, e.g., prostate cancer, ovarian cancer, or hepatocellular carcinoma (HCC). Diagnosis may be performed by any method or techniques known in the art, such as x-ray, MRI, or biopsy, and confirmed by a physician. To minimize exposure of a patient to drug treatments that may not be therapeutic, the patient may be determined to be either responsive or non-responsive to a cancer treatment, such as 2,2′-dithio-bis-ethane sulfonate, according to the methods described herein. In certain examples, the sample is selected from the group consisting of tissue, blood, plasma, serum, urine, urine supernatant, a urine cell pellet, semen, prostatic secretions and prostate cells.

Another embodiment includes a method for treating or determining the sensitivity of non-small cell cancer to a 2,2′-dithio-bis-ethane sulfonate treatment by assessing the level of NRF2 (SEQ ID NO: 1) expression and at least one gene selected from the group consisting of: NQO1 (SEQ ID NO: 2), PHGDH (SEQ ID NO: 3), HMOX1 (SEQ ID NO: 4), SLC7A11 (SEQ ID NO: 5), SRXN1 (SEQ ID NO: 6), SOX2(SEQ ID NO: 7), GPX2 (SEQ ID NO: 8), GPX3 (SEQ ID NO: 9), and GPX7(SEQ ID NO: 10). For illustration, non-small cell lung cancer cell line HCC827 was treated for 2 h with 2,2′-dithio-bis-ethane sulfonate alone at concentrations of 1 mM and 15 mM and gene expression level changes were measured by whole transcriptome profiling using RNAseq. A control sample with no treatment for the same duration was used as the baseline gene expression level. A greater than 1.5 to 2-fold expression level change can be indicative of 2, 2′-dithio-bis-ethane sulfonate sensitivity.

In another embodiment, NRF2 (SEQ ID NO: 1) can be an alias of NFE2L2.

In another embodiment, the detection of NRF2 in connection with one or more of the following genes: JAG1, IGF1, NANOG, GPX6, GPXS, PRDX4, TXN, TERT, TXNRD2, FGF2, PRDX2, PRDX3, PRDX1, BMPR1A, PDGFC, GPX1, PSPH, SHMT1, TXN2, SHMT2, PRDX6, NPNT, PSAT1, NOTCH1, GLRX2, VEGFC, GSR, ADAM10, PRDXS, TXNRD1, GLRX, ATF4, SIRT1, ITGB2, G6PD, GPX4, GPX3, GPX2, NQO1, PHGDH, GPX7, SLC7A11, SOX2, SRXN1, HMOX1, BCL2, BMI1, HEBP1, NFE2L2, PGK1, POU5F1, and TALDO1.

One embodiment includes a method of treating cancer in a patient in need thereof that includes administering 2,2′-dithio-bis-ethane sulfonate to the patient, in which the patient has been determined to be responsive to 2,2′-dithio-bis-ethane sulfonate according to the method disclosed herein. In particular, the patient may have a cancer that is resistant to one or more cancer therapies other than or together with 2,2′-dithio-bis-ethane sulfonate (e.g., a patient with prostate cancer, ovarian cancer, or HCC that is resistant to one or more cancer therapies other than or together with 2,2′-dithio-bis-ethane sulfonate).

Another embodiment includes a method of treating solid tumor cancer in a subject, comprising: (a) obtaining or having obtained an expression level in a sample from a subject for a plurality of biomarkers, wherein the plurality of biomarkers comprises (1) NRF2 (SEQ ID NO: 1) and at least one biomarker selected from the group consisting of NQO1 (SEQ ID NO: 2), PHGDH (SEQ ID NO: 3), HMOX1 (SEQ ID NO: 4), SLC7A11 (SEQ ID NO: 5), SRXN1 (SEQ ID NO: 6), SOX2(SEQ ID NO: 7), GPX2 (SEQ ID NO: 8), GPX3 (SEQ ID NO: 9), and GPX7(SEQ ID NO: 10; (b) determining that the subject is sensitive to a treatment with 2,2′-dithio-bis-ethane sulfonate; and (c) administering a cancer treatment including 2,2′-dithio-bis-ethane sulfonate.

In one embodiment, the patient or subject is treated with 2,2′-dithio-bis-ethane sulfonate together with one or more other cancer treatments. For example, such treatments include cisplatin, paclitaxel, and other available therapies.

Methods include administering to a subject who is receiving or will receive a chemotherapeutic agent, an effective amount of 2,2′-dithio-bis-ethane sulfonate, a pharmaceutically-acceptable salt thereof, and/or an analog thereof, administered at a rate of about 0.1 g/min to about 2.0 g/min to the subject.

In another embodiment, an effective amount of 2,2′-dithio-bis-ethane sulfonate, a pharmaceutically-acceptable salt thereof, and/or an analog thereof, is administered at a rate of about 0.2 g/min to about 1.0 g/min to the subject.

In another embodiment, an effective amount of 2,2′-dithio-bis-ethane sulfonate, a pharmaceutically-acceptable salt thereof, and/or an analog thereof, is administered at a rate of about 0.7 g/min to the subject.

In one embodiment, a dose of 2,2′-dithio-bis-ethane sulfonate, a pharmaceutically-acceptable salt thereof, and/or an analog thereof, is administered over a period of about 45 minutes to the subject.

In another embodiment, the total dose of 2,2′-dithio-bis-ethane sulfonate, a pharmaceutically-acceptable salt thereof, and/or an analog thereof, administered to a subject is from about 4.0 g/m² to about 35 g/m ². One dose is about 18.4 g/m². The administration of one or more of said doses of the compounds to a subject can be over about 45 minutes.

The invention also includes methods of reducing, preventing, mitigating, delaying the onset of, attenuating the severity of, and/or hastening the resolution of chemotherapy-associated toxicity in a subject receiving a chemotherapy agent, comprising administering to the subject an effective amount of 2,2′-dithio-bis-ethane sulfonate, a pharmaceutically-acceptable salt thereof, and/or an analog thereof, at a rate of about 0.1 g/min to about 4.6 g/min, at a total dose of about 4 g/m² to about 35 g/m². Preferred is administration of a total dose of about 18.4 g/m² at a rate of about 0.1 g/min to about 4.6 g/min to a subject. Particularly preferred is administration of a total dose of about 18.4 g/m² over about 45 minutes to a subject at an administration rate of about 0.4 g/m²/min.

In certain examples, the methods disclosed herein can further comprise using a machine to isolate the biomarker or the probe from the sample. Alternatively, or additionally, the methods disclosed herein further comprise contacting the sample with a label that specifically binds to the biomarkers, the probe, or a combination thereof. In some embodiments, the methods disclosed herein further comprise contacting the sample with a label that specifically binds to a biomarker selected from genes herein. In some embodiments, the methods disclosed herein further comprise amplifying the biomarker, the probe, or any combination thereof. The methods disclosed herein can further comprise sequencing the target, the probe, or any combination thereof. In some instances, the method further comprises quantifying the expression level of the plurality of biomarkers. In some embodiments, the method further comprises labeling the plurality of biomarkers.

In some embodiments, diagnosing, predicting, and/or monitoring the status or outcome of a cancer may comprise determining a therapeutic 2,2′-dithio-bis-ethane sulfonate regimen. Determining a therapeutic regimen may comprise administering an anti-cancer therapeutic. Alternatively, determining the treatment for the cancer may comprise modifying a therapeutic regimen to include or reduce 2,2′-dithio-bis-ethane sulfonate. Modifying a therapeutic regimen may comprise increasing, decreasing, or terminating a therapeutic regimen.

In another embodiment, the 2,2′-dithio-bis-ethane sulfonate is a disodium salt.

In other embodiments, the 2,2′-dithio-bis-ethane sulfonate can be analogs including, for example, monosodium 2,2′-dithio-bis-ethane sulfonate, sodium potassium 2,2′-dithio-bis-ethane sulfonate, dipotassium 2,2′-dithio-bis-ethane sulfonate, calcium 2,2′-dithio-bis-ethane sulfonate, magnesium 2,2′-dithio-bis-ethane sulfonate, monopotassium 2,2′-dithio-bis-ethane sulfonate, or manganese 2,2′-dithio-bis-ethane sulfonate; ammonium 2,2′-dithio-bis-ethane sulfonate.

In certain embodiments, 2,2′-dithio-bis-ethane sulfonate is administered with a chemotherapeutic agent that is, for example: a fluoropyrimidine; a pyrimidine nucleoside; a purine nucleoside; an antifolate, a platinum analog; an anthracycline/anthracenedione; an epipodophyllotoxin; a camptothecin; a hormone, a hormonal analog; an antihormonal; an enzyme, protein, peptide, or polyclonal and monoclonal antibody; a vinca alkaloid; a taxane; an epothilone; an antimicrotubule agent; an alkylating agent; an antimetabolite; a topoisomerase inhibitor; an antiviral; or another cytotoxic and/or cytostatic agent. Fluoropyrimidines include, for example, 5-fluorouracil (5-FU), S-1 capecitabine, ftorafur, 5′deoxyfluorouridine, UFT, eniluracil, and the like. Pyrimidine nucleosides include, for example, cytarabine, deoxycytidine, 5-azacytosine, gemcitabine, 5-azacytosine, 5-azadeoxycytidine, and the like. Purine nucleosides include, for example, fludarabine, 6-mercaptopurine, thioguanine, allopurinol, cladribine, and 2-chloro adenosine. Antifolates include, for example, methotrexate (MTX), trimetrexate, aminopterin, and methylene-10-deazaminopterin (MDAM). Platinum analogs include, for example, cisplatin, carboplatin, oxaliplatin, satraplatin, picoplatin, tetraplatin, platinum-DACH and analogs thereof. Anthracyclines/anthracenediones include, for example, doxorubicin, daunorubicin, epirubicin, and idarubicin. Epipodophyllotoxin derivatives include, for example, etoposide, etoposide phosphate and teniposide. Camptothecins include, for example, irinotecan, topotecan, 9-aminocamptothecin, 10,11-methylenedioxycamptothecin, karenitecin, 9-nitrocamptothecin, and TAS 103. Hormones and hormonal analogs may include, for example, estrogens and estrogen analogs, including anastrazole, diethylstilbesterol, estradiol, premarin, raloxifene; progesterone, progesterone analogs and progestins, including progesterone, norethynodrel, esthisterone, dimesthisterone, megestrol acetate, medroxyprogesterone acetate, hydroxyprogesterone caproate, and norethisterone; androgens, including fluoxymesterone, methyltestosterone and testosterone; as well as adrenocorticosteroids, including dexamthasone, prednisone, cortisol, solumedrol, and the like. Antihormones include, for example, (i) antiestrogens, including: tamoxifen, fulvestrant, toremifene; aminoglutethimide, testolactone, droloxifene, anastrozole; (ii) antiandrognes, including: bicalutamide, flutamide, nilutamide, goserelin; (iii) antitestosterones, including: flutamide, leuprolide, and triptorelin; (iv) adrenal steroid inhibitors including: aminoglutethimide and mitotane; and anti-leuteinizing hormones, including goserelin. Enzymes, proteins, peptides, polyclonal and/or monoclonal antibodies, may include, for example, asparaginase, cetuximab, erlotinib, bevacizumab, rituximab, gefitinib, trastuzumab, interleukins, interferons, leuprolide, pegasparanase, and the like. Vinca Alkaloids include, for example, vincristine, vinblastine, vinorelbine, vindesine, and the like. Taxanes include, for example, paclitaxel, docetaxel, and formulations and analogs thereof Alkylating agents may include, for example, dacarbazine; procarbazine; temozolamide; thiotepa; nitrogen mustards (e.g., mechlorethamine, chlorambucil, L-phenylalanine mustard, melphalan, and the like); oxazaphosphorines (e.g., ifosphamide, cyclophosphamide, mefosphamide, perfosfamide, trophosphamide and the like); alkyl sulfonates (e.g., busulfan); and nitrosoureas (e.g., carmustine, lomustine, semustine and the like). Epothilones include, for example, epothilones A-E. Antimetabolites include, for example, tomudex and methotrexate, 6-mercaptopurine, and 6-thioguanine. Topoisomerase inhibitors include, for example, irinotecan, and topotecan, karenitecin, amsacrine, etoposide, etoposide phosphate, teniposide, and doxorubicin, daunorubicin, and other analogs. Antiviral agents include, for example, acyclovir, valacyclovir, ganciclovir, amantadine, rimantadine, lamivudine, and zidovudine. Monoclonal antibody agents include, for example, bevacizumab, trastuzumab, rituximab, and the like, as well as growth inhibitors such as erlotinib, and the like. In general, cytostatic agents are mechanism-based agents that slow the progression of neoplastic disease.

Detecting Biomarker for 2,2′-dithio-bis-ethane Sulfonate Treatment Sensitivity

It will be apparent from the preceding description that the diagnostic methods of the present invention may involve a degree of quantification to determine levels biomarkers in patient samples. Such quantification is readily provided by the inclusion of appropriate control samples.

In one embodiment, internal controls are included in the methods of the present invention. A preferred internal control is one or more samples taken from one or more healthy individuals.

As will be known to those skilled in the art, when internal controls are not included in each assay conducted, the control may be derived from an established data set.

Data pertaining to the control subjects can selected from the group consisting of: 1. a data set comprising measurements of the presence or level of expression of biomarkers for a typical population of subjects known to have cancer or specific cancer; 2. a data set comprising measurements of the presence or level of biomarkers for the subject being tested wherein said measurements have been made previously, such as, for example, when the subject was known to be healthy or, in the case of a subject having cancer, when the subject was diagnosed or at an earlier stage in disease progression; 3. a data set comprising measurements of the presence or level of biomarkers for a healthy individual or a population of healthy individuals; and 4. a data set comprising measurements of the presence or level of biomarkers for a normal individual or a population of normal individuals.

Those skilled in the art are readily capable of determining the baseline for comparison in any diagnostic assay of the present invention without undue experimentation, based upon the teaching provided herein.

Compounds that bind a biomarker when used diagnostically may be linked to a diagnostic reagent such as a detectable label to allow easy detection of binding events in vitro or in vivo. Suitable labels include radioisotopes, dye markers or other imaging reagents for detection and/or localization of target molecules. Compounds linked to a detectable label can be used with suitable in vivo imaging technologies such as, for example, radiology, fluoroscopy, nuclear magnetic resonance imaging (MRI), CAT-scanning, positron emission tomography (PET), computerized tomography etc.

Specific methods are able to detect sensitivity to a cancer treatment comprising 2,2′-dithio-bis-ethane sulfonate. As would be understood by the person skilled in the art, sensitivity refers to the proportion of actual positives in the test which are correctly identified as having sensitivity to a treatment comprising 2,2′-dithio-bis-ethane sulfonate or analogs. In one embodiment, the methods of the invention are able to detect 2,2′-dithio-bis-ethane sulfonate with a sensitivity of at least 50%, 60% or 66%, or at least 75%, 80%, 85%,88%, 89%, 90%, or at least 95%. In another embodiment, the methods of the invention are able to diagnose or detect cancer with a sensitivity of at least 80%, or at least 85% or at least 90%, or at least 95%.

### Protein Detection Techniques

In one embodiment, biomarker polypeptide is detected in a patient sample, wherein the presence and/or level of the polypeptide in the sample is indicative of cancer. For example, the method may comprise contacting a biological sample derived from the subject with a compound capable of binding to a biomarker polypeptide, and detecting the formation of complex between the compound and the biomarker polypeptide. The term “biomarker polypeptide” as used herein includes fragments of biomarker polypeptides, including for example, immunogenic fragments and epitopes of the biomarker polypeptide.

In one embodiment, the compound that binds the biomarker is an antibody.

In another embodiment, an antibody to a biomarker polypeptide is detected in a patient sample, wherein the presence and/or level of the antibody in the sample is indicative of cancer.

Preferred detection systems contemplated herein include any known assay for detecting proteins or antibodies in a biological sample isolated from a human subject, such as, for example, SDS/PAGE, isoelectric focusing, 2-dimensional gel electrophoresis comprising SDS/PAGE and isoelectric focusing, an immunoassay, flow cytometry e.g. fluorescence-activated cell sorting (FACS), a detection based system using an antibody or non-antibody compound, such as, for example, a small molecule (e.g. a chemical compound, agonist, antagonist, allosteric modulator, competitive inhibitor, or non-competitive inhibitor, of the protein). In accordance with these embodiments, the antibody or small molecule may be used in any standard solid phase or solution phase assay format amenable to the detection of proteins. Optical or fluorescent detection, such as, for example, using mass spectrometry, MALDI-TOF, biosensor technology, evanescent fiber optics, or fluorescence resonance energy transfer, is clearly encompassed by the present invention. Assay systems suitable for use in high throughput screening of mass samples, e.g. a high throughput spectroscopy resonance method (e.g. MALDI-TOF, electrospray MS or nano-electrospray MS), are also contemplated. Another suitable protein detection technique involves the use of Multiple Reaction Monitoring (MRM) in LC-MS (LC/MRM-MS) (Anderson and Hunter, 2006).

Immunoassay formats are particularly suitable, e.g., selected from the group consisting of, an immunoblot, a Western blot, a dot blot, an enzyme linked immunosorbent assay (ELISA), radioimmunoassay (MA), enzyme immunoassay. Modified immunoassays utilizing fluorescence resonance energy transfer (FRET), isotope-coded affinity tags (ICAT), matrix-assisted laser desorption/ionization time of flight (MALDI-TOF), electrospray ionization (ESI), biosensor technology, evanescent fiber-optics technology or protein chip technology are also useful.

### Nucleic Acid Detection Techniques

Any suitable technique that allows for the qualitative and/or quantitative assessment of the level of a biomarker polynucleotide in a sample may be used. The terms “nucleic acid molecule” or “polynucleotide” as used herein refer to an oligonucleotide, polynucleotide or any fragment thereof.

Comparison may be made by reference to a standard control, or to a control level that is found in healthy tissue. For example, levels of a transcribed gene can be determined by Northern blotting, and/or RT-PCR. With the advent of quantitative (real-time) PCR, quantitative analysis of gene expression can be achieved by using appropriate primers for the gene of interest. The nucleic acid may be labelled and hybridized on a gene array, in which case the gene concentration will be directly proportional to the intensity of the radioactive or fluorescent signal generated in the array.

Methods for direct sequencing of nucleotide sequences are well known to those skilled in the art and can be found for example in Ausubel et al., eds., Short Protocols in Molecular Biology, 3rd ed., Wiley, (1995) and Sambrook et al., Molecular Cloning, 3rd ed., Cold Spring Harbor Laboratory Press, (2001). Sequencing can be carried out by any suitable method, for example, dideoxy sequencing, chemical sequencing or variations thereof. Direct sequencing has the advantage of determining variation in any base pair of a particular sequence.

The nucleic acid may be separated from the sample for testing. Suitable methods will be known to those of skill in the art. For example, RNA may be isolated from a sample to be analysed using conventional procedures, such as are supplied by QIAGEN technology. This RNA is then reverse-transcribed into DNA using reverse transcriptase and the DNA molecule of interest may then be amplified by PCR techniques using specific primers.

Diagnostic procedures may also be performed directly upon patient samples. Hybridisation or amplification assays, such as, for example, Southern or Northern blot analysis, immunohistochemistly, single-stranded conformational polymorphism analysis (SSCP) and PCR analyses are among techniques that are useful in this respect. If desired, target or probe nucleic acid may be immobilised to a solid support such as a microtitre plate, membrane, polystyrene bead, glass slide or other solid phase.

### Kits

The present invention provides kits for the diagnosis or detection of cancer. Such kits may be suitable for detection of nucleic acid species, or alternatively may be for detection of a polypeptide gene product, as discussed above.

For detection of polypeptides, antibodies will most typically be used as components of kits. However, any agent capable of binding specifically to a biomarker gene product will be useful in this aspect of the application. Other components of the kits will typically include labels, secondary antibodies, substrates (if the gene is an enzyme), inhibitors, co-factors and control gene product preparations to allow the user to quantitate expression levels and/or to assess whether the diagnosis experiment has worked correctly. Enzyme-linked immunosorbent assay-based (ELISA) tests and competitive ELISA tests are particularly suitable assays that can be carried out easily by the skilled person using kit components.

Optionally, the kit further comprises means for the detection of the binding of an antibody to a biomarker polypeptide. Such means include a reporter molecule such as, for example, an enzyme (such as horseradish peroxidase or alkaline phosphatase), a dye, a radionucleotide, a luminescent group, a fluorescent group, biotin or a colloidal particle, such as colloidal gold or selenium. Preferably such a reporter molecule is directly linked to the antibody.

In yet another embodiment, a kit may additionally comprise a reference sample. In one embodiment, a reference sample comprises a polypeptide that is detected by an antibody. Preferably, the polypeptide is of known concentration. Such a polypeptide is of particular use as a standard. Accordingly, various known concentrations of such a polypeptide may be detected using a diagnostic assay described herein.

For detection of nucleic acids, such kits may contain a first container such as a vial or plastic tube or a microtiter plate that contains an oligonucleotide probe. The kits may optionally contain a second container that holds primers. The probe may be hybridisable to DNA whose altered expression is associated with cancer and the primers are useful for amplifying this DNA. Kits that contain an oligonucleotide probe immobilised on a solid support could also be developed, for example, using arrays (see supplement of issue 21(1) Nature Genetics, 1999).

For PCR amplification of nucleic acid, nucleic acid primers may be included in the kit that are complementary to at least a portion of a biomarker gene as described herein. The set of primers typically includes at least two oligonucleotides, preferably four oligonucleotides, that are capable of specific amplification of DNA. Fluorescent-labelled oligonucleotides that will allow quantitative PCR determination may be included (e.g. TaqMan chemistry, Molecular Beacons). Suitable enzymes for amplification of the DNA, will also be included.

### Regression Algorithms and Statistics

In order to develop a panel of biomarkers suitable for diagnosing or detecting cancer, the present inventors have analyzed numerous biomarkers in a statistical model. Such an improvement in the performance of a test is sometimes referred to as the “in-sample” performance. A fair evaluation of a test requires its assessment using out-of-sample subjects, that is, subjects not included in the construction of the initial predictive model. This is achieved by assessing the test performance using cross validation.

Tests for statistical significance include linear and nonlinear regression, including ANOVA, Kruskal-Wallis, Wilcoxon, Mann-Whitney and odds ratio, Baysian probability algorithms. As the number of biomarkers measured increases however, it can be generally more convenient to use a more sophisticated technique such as Random Forests, simple logistic, Bayes Net to name a few.

For example, Bayesian probability may be adopted. In this circumstance a 10-fold cross-validation can be used to estimate the “out-of-sample” performance of the models in question. For each combination of biomarkers under consideration, the data can be divided randomly into 10 sub-samples, each with similar proportions of healthy subject and subjects at each stage of disease. In turn, each subsample can be excluded, and a logistic model built using the remaining 90% of the subjects. This model can then be used to estimate the probability of cancer for the excluded sub-sample, providing an estimate of “out-of-sample” performance. By repeating this for the remaining 9 sub samples, “out-of-sample” performance can be estimated from the study data itself. These out-of sample predicted probabilities can then be compared with the actual disease status of the subjects to create a Receiver Operating Characteristic (ROC) Curve, from which the cross-validated sensitivity at 95% specificity may be estimated.

Each estimate of “out-of-sample” performance using cross-validation (or any other method), whilst unbiased, has an element of variability to it. Hence a ranking of models (based on biomarker combinations) can be indicative only of the relative performance of such models. However a set of biomarkers which is capable of being used in a large number of combinations to generate a diagnostic test as demonstrated via “out-of-sample” performance evaluations, almost certainly contains within itself combinations of biomarkers that will withstand repeated evaluation.

Thus, in light of the teachings of the present specification, the person skilled in the art will appreciate that the sensitivity and specificity of a test for diagnosing cancer may be modulated by selecting a different combination of the biomarkers as described herein

### EXAMPLE

### Example 1

Non-small cell lung cancer cell line HCC827 was treated for 2 h with disodium 2,2′-dithio-bis-ethane sulfonate alone at concentrations of 1 mM and 15 mM and gene expression level changes were measured by whole transcriptome profiling using RNAseq. A control sample with no treatment for the same duration was used as the baseline gene expression level. Using a threshold of fold change of the biomarker genes as well as NRF2 itself, nine genes are upregulated genes in response to disodium 2,2′-dithio-bis-ethane sulfonate exposure. These 9 genes include NQO1 (SEQ ID NO: 2), PHGDH (SEQ ID NO: 3), HMOX1 (SEQ ID NO: 4), SLC7A11 (SEQ ID NO: 5), SRXN1 (SEQ ID NO: 6), SOX2(SEQ ID NO: 7), GPX2 (SEQ ID NO: 8), GPX3 (SEQ ID NO: 9), and GPX7(SEQ ID NO: 10. A greater than 1.5 to 2-fold expression level change from the reference level was be indicative of disodium 2, 2′-dithio-bis-ethane sulfonate sensitivity

### Example 2

A showed the induction of nuclear expression of nuclear factor erythroid 2-related factor 2 (Nrf2) by 2,2′-dithio-bis-ethane sulfonate in PC12 cells (rat adrenal medulla pheochromocytoma cell line) differentiated into neuron-like cells by nerve growth factor (NGF) was examined. PC12 cells were suspended in DMEM containing 7.5% inactivated fetal bovine serum and 7.5% inactivated horse serum. After seeding onto a type I collagen-coated 100 mm culture dish, these cells were incubated for 7 days in the presence of 100 ng/mL NGF for priming. Culture medium was then replaced with Neurobasal™ medium supplemented with N-2 supplement and 2 mM glutamine containing NGF 30 ng/mL, and cells were incubated again for 3 days for differentiation into neuron-like cells. Next, these cells were treated with 2,2′-dithio-bis-ethane sulfonate 6 mM for 1, 2, 4 and 8 hours, or with a known Nrf2 activator, tert-butylhydroquinone (t-BHQ) 30 μM for 3 hours. Nuclear proteins were then extracted from the cells for detection of nuclear expression of Nrf2 by western blotting.

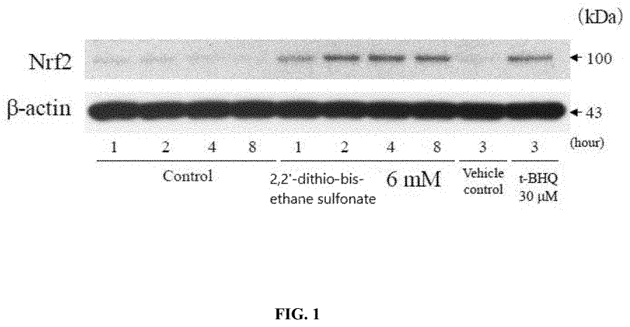

FIG. 1 shows the induction of nuclear expression of Nrf2 in cells treated with t-BHQ 30 μM and cells treated with 2,2′-dithio-bis-ethane sulfonate. 6 mM, nuclear expression of Nrf2 started at 1 hour after the start of treatment, and apparent expression of Nrf2 persisted until 8 hours after the start of treatment. In control cells treated with 2,2′-dithio-bis-ethane sulfonate or t-BHQ alone, nuclear expression of Nrf2 was not generally observed at any treatment duration.

These results revealed that in PC12 cells differentiated into neuron-like cells, 2,2′-dithio-bis-ethane sulfonate shows nuclear expression of Nrf2 indicates sensitivity to 2,2′-dithio-bis-ethane sulfonate.

1. A method for treating cancer in a patient, comprising
(a) obtaining a sample of the cancer from the patient,
(b) treating the sample with a 2,2′-dithio-bis-ethane sulfonate analog;
(c) obtaining an expression level in the sample for one or more of a plurality of biomarkers, wherein the plurality of biomarkers includes NRF2 (SEQ ID NO: 1);
(d) determining that the sample is sensitive to a treatment with disodium 2,2′-dithio-bis-ethane sulfonate from the expression levels of NRF2 (SEQ ID NO: 1); and
(e) administering a cancer treatment to the subject including a disodium 2,2′-dithio-bis-ethane sulfonate analog if the expression level of NRF2 (SEQ ID NO: 1) is greater than 1 times compared to non-cancer cells.

2. The method of claim 1, wherein the plurality of biomarkers further includes one biomarker selected from the group consisting of NQO1 (SEQ ID NO: 2), PHGDH (SEQ ID NO: 3), HMOX1 (SEQ ID NO: 4), SLC7A11 (SEQ ID NO: 5), SRXN1 (SEQ ID NO: 6), SOX2(SEQ ID NO: 7), GPX2 (SEQ ID NO: 8), GPX3 (SEQ ID NO: 9), and GPX7 (SEQ ID NO: 10).

3. The method of claim 1, wherein the non-cancer cells are the level of the expression in the biological sample obtained from a healthy patient and are the reference level.

4. The method of claim 1, wherein the 2,2′-dithio-bis-ethane sulfonate analog is disodium 2,2′-dithio-bis-ethane sulfonate.

5. The method of claim 1, wherein the cancer treatments comprise the administration of one or more chemotherapy agents selected from the group consisting of: platinum complexes and taxanes.

6. The method of claim 1, wherein the cancer treatment includes radiation treatment.

7. The method of claim 1, wherein one or more nucleic acid molecules from the sample are contacted with a device comprising (a) a first single-stranded nucleic acid molecules capable of specifically hybridizing with the nucleotides of the biomarkers; and (b) a second single-stranded nucleic acid molecules capable of specifically hybridizing with the nucleotides of a plurality of biomarkers, wherein the biomarkers are nucleic acid.

8. A method of treating solid tumor cancer in a subject, comprising:
(a) treating the tumor with 2,2′-dithio-bis-ethane sulfonate analog;
(b) obtaining an expression level in a sample from a subject one of a plurality of biomarkers, wherein the plurality of biomarkers comprises NRF2(SEQ ID NO: 1);
(c) determining that the tumor is sensitive to a treatment with disodium 2,2′-dithio-bis-ethane sulfonate analog from the expression levels of NRF2(SEQ ID NO: 1); and
(d) administering a cancer treatment including disodium 2,2′-dithio-bis-ethane sulfonate if the expression level of NRF2(SEQ ID NO: 1) is greater than 1 times the non-cancer cells.

9. The method of claim 8, wherein the plurality of biomarkers also includes and one biomarker selected from the group consisting of NQO1 (SEQ ID NO: 2), PHGDH (SEQ ID NO: 3), HMOX1 (SEQ ID NO: 4), SLC7A11 (SEQ ID NO: 5), SRXN1 (SEQ ID NO: 6), SOX2(SEQ ID NO: 7), GPX2 (SEQ ID NO: 8), GPX3 (SEQ ID NO: 9), and GPX7(SEQ ID NO: 10).

10. The method of claim 8, wherein the compound is disodium 2,2′-dithio-bis-ethane sulfonate analog.

11. The method of claim 9, wherein the compound ranged from approximately 14 g/m² to approximately 22 g/m².

12. The method of claim 10, wherein said chemotherapy treatments comprise the administration of one or more chemotherapy agents selected from the group consisting of:
platinum complexes and taxanes.

13. The method of claim 11, wherein said platinum complex medicaments are selected from the group consisting of: cisplatin, oxaliplatin, carboplatin, satraplatin, and derivatives and analogs thereof.

14. A method of testing a tumor sample of a patient having a known cancer type, wherein the patient is resistant to one or more cancer therapies and has an unknown responsiveness to disodium 2,2′-dithio-bis-ethane sulfonate, comprising:
contacting the sample with disodium 2,2′-dithio-bis-ethane sulfonate;
contacting the sample with a device comprising (a) a first single-stranded nucleic acid molecules capable of specifically hybridizing with the nucleotides of a plurality of biomarkers, wherein a first biomarker is NFR2; and (b) a second single-stranded nucleic acid molecules capable of specifically hybridizing with the nucleotides of a plurality of biomarkers of resistance selected from the biomarkers of NQO1 (SEQ ID NO: 2), PHGDH (SEQ ID NO: 3), HMOX1 (SEQ ID NO: 4), SLC7A11 (SEQ ID NO: 5), SRXN1 (SEQ ID NO: 6), SOX2(SEQ ID NO: 7), GPX2 (SEQ ID NO: 8), GPX3 (SEQ ID NO: 9), and GPX7(SEQ ID NO: 10);
detecting a level of expression of the plurality of biomarkers; and
administering disodium 2,2′-dithio-bis-ethane sulfonate to the patient, wherein the patient has been determined to be responsive to disodium 2,2′-dithio-bis-ethane sulfonate.

15. A greater than 1.5 to 2-fold expression level change can be indicative of 2, 2′-dithio-bis-ethane sulfonate sensitivity
